(19) United States
(12) Patent Application Publication
(10) Pub. No.: US 2003/0167526 A1
(43) Pub. Date: Sep. 4, 2003

(54) Compositions and methods for identifying transformed cells

(75) Inventors: Keith S. Lowe, Johnston, IA (US); William J. Gordon-Kamm, Urbandale, IA (US); George J. Hoerster, Des Moines, IA (US)

Correspondence Address: PIONEER HI-BRED INTERNATIONAL INC., 7100 N.W. 62ND AVENUE, P.O. BOX 1000, JOHNSTON, IA, 50131, US

(73) Assignee: Pioneer Hi-Bred International Inc.

(21) Appl. No.: 10/337,562

(22) Filed: Jan. 7, 2003

Related U.S. Application Data
(60) Provisional application No. 60/348,438, filed on Jan. 14, 2002.

(51) Int. Cl. A01H 1/00; C12N 15/82; C12Q 1/00

(52) U.S. Cl. 800/282; 435/4

(57) ABSTRACT

The present invention relates to compositions and methods for identifying transformed cells. The method comprises introducing a visual marker polynucleotide into a plant cell and providing a growth stimulation protein. The compositions comprise a visual marker polynucleotide and a growth stimulation polynucleotide. Also provided are expression cassettes, plant cells, plant parts, and plants comprising same.

### CROSS REFERENCE TO RELATED APPLICATIONS

This application claims priority to U.S. Serial No. 60/348,438 filed Jan. 14, 2002, the disclosure of which is incorporated herein by reference.

### FIELD OF THE INVENTION

The present invention relates generally to plant molecular biology. More specifically, it relates to identification of transgenic plant cells.

### BACKGROUND OF THE INVENTION

An important aspect of plant transformation is the ability to screen for transformed plants. Various selectable marker genes that provide lethal selection have been used. Unfortunately use of the selection media is often harmful to the transformed cells containing the selectable marker gene.

Regulatory genes activating the anthocyanin pathway were cloned in the mid-eighties. These genes were found to be efficient markers for identification of transformed cells (see WO 91/02059) and Ludwig et al (Ludwig, S. R., Bowen, B., Beach, I., and Wessler, S. R. 1990 Science 247:449-450). Although these genes worked well as markers for transient expression, toxicity of the anthocyanin proteins reduced transformation frequencies, making it difficult to obtain transformed plants and progeny using visual selection alone (Bower et al., 1996; Chawla et al., 1999). Therefore, it would be valuable to develop a method for screening transformed plant cells that avoids these problems.

### SUMMARY OF THE INVENTION

The present invention relates to compositions and methods for identifying transformed cells. The method comprises introducing a visual marker polynucleotide into a plant cell and providing a growth stimulation protein. The compositions comprise a visual marker polynucleotide and a growth stimulation polynucleotide. Also provided are expression cassettes, plant cells, plant parts, and plants comprising same.

### DETAILED DESCRIPTION OF THE INVENTION

Definitions

The term “isolated” refers to material, such as a nucleic acid or a protein, which is: (1) substantially or essentially free from components which normally accompany or interact with the material as found in its naturally occurring environment or (2) if the material is in its natural environment, the material has been altered by deliberate human intervention to a composition and/or placed at a locus in the cell other than the locus native to the material.

As used herein, “nucleic acid or polynucleotide” means a polynucleotide and includes single or double-stranded polymer of deoxyribonucleotide or ribonucleotide bases. Nucleic acids may also include modified nucleotides that permit read through by a polymerase.

As used herein, “polypeptide” means proteins, protein fragments, modified proteins, amino acid sequences and synthetic amino acid sequences. The polypeptide can be glycosylated or not.

As used herein, “anthocyanin polynucleotide” means a nucleic acid or polynucleotide that codes for an anthocyanin polypeptide.

As used herein, “complementary anthocyanin polynucleotides” means nucleic acids or polynucleotides that code for complementary anthocyanin polypeptides.

As used herein, “anthocyanin polypeptide” means a polypeptide or combination of polypeptides that activate or are involved in anthocyanin biosynthesis.

As used herein, “complementary anthocyanin polypeptides” means two or more anthocyanin polypeptides that when combined result in anthocyanin accumulation. For example a member of the C1 family and a member of the R family.

As used herein, “fluorescent proteins” are proteins that absorb UV or visible light radiation and emit visible radiation at a higher wavelength.

As used herein, “growth stimulation polynucleotide” means a polynucleotide that alters or modulates the activity of a polypeptide to stimulate growth of a cell. The activity of the polypeptide can be increased or decreased as needed to stimulate growth of the cell.

As used herein, “growth stimulation polypeptide” means a polypeptide capable of influencing growth of a cell.

As used herein, “LEC1 polynucleotide” means a nucleic acid or polynucleotide that codes for a LEC1 polypeptide.

As used herein, “LEC1 polypeptide” means a HAP3 family member, CCAAT-box binding transcriptional activator polypeptide that regulates gene expression during embryo development. A LEC1 polypeptide is a growth stimulation polypeptide.

As used herein, “plant” includes plants and plant parts including but not limited to plant cells, plant tissue such as leaves, stems, roots, flowers, embryos, and seeds.

As used herein, “promoter” includes reference to a region of DNA upstream from the start of transcription and involved in recognition and binding of RNA polymerase and other proteins to initiate transcription.

The present invention provides compositions and methods for identifying transformed plant cells. The method comprises introducing a visual marker polynucleotide into a plant cell and providing a growth stimulation protein. The compositions comprise a visual marker polynucleotide and a growth stimulation polynucleotide. Also provided are expression cassettes, plant cells, plant parts, and plants comprising same.

Various visual markers are known in the art. For example fluorescent proteins available from Aurora or Clonetech, Palo Alto, Calif. are useful visual markers. Fluorescent protein genes are described in various patents such as PCT publication WO 97/41228 (monocot codon optimized); U.S. Pat. No. 5,985,577; U.S. Pat. Nos. 5,625,048; 5,777,079; U.S. Pat. No. 5,491,084 green fluorescent protein (GFP); U.S. Pat. No. 6,146,826 mutant GFP; and U.S. Pat. No. 5,741,668 mutant GFP. Luciferase is described in several patents, U.S. Pat. No. 5,418,155; U.S. Pat. No. 5,292,658; U.S. Pat. No. 5,583,024; U.S. Pat. No. 5,674,713; U.S. Pat. Nos. 5,700,673; 5,283,179; and PCT application WO 93/01283. Luciferase genes are available from Promega, Madison, Wis. These markers can be introduced with various promoters described below.

Other visual markers include anthocyanin pigments. Many plant cell types have the capacity to accumulate anthocyanin pigments. In the following discussion, maize plants will be used as an example for illustrative purposes. However, the present invention relates to all plant cell types that can accumulate anthocyanin pigments under the control of anthocyanin polypeptides.

At least five genes in maize are known to encode enzymes that are required for the synthesis of anthocyanin pigments. Several other loci, including R, B, and Lc, determine the pattern and timing of anthocyanin biosynthesis in the maize plant and seed. Genes from the maize R gene complex encode a protein that acts to regulate the production of anthocyanin pigments in most seed and plant tissue. Maize strains can have one, or as many as four, R alleles which combine to regulate pigmentation in a developmental and tissue specific manner. Thus, an R gene introduced into such cells will cause the expression of a red pigment and, if stably incorporated, can be visually scored as a red sector. If a maize line carries dominant alleles for genes encoding for enzymatic intermediates in the anthocyanin biosynthetic pathway (C2, A1, A2, Bz1 and Bz2), but carries a recessive allele at the R locus, transformation of any cell from that line with R will result in red pigment formation.

Various monocot plants can be utilized if the C1 and R alleles are introduced together. By controlling expression of the C1 and R alleles it is possible to tightly regulate expression in genotypes where the endogenous alleles are recessive. For example, the combination of a tissue specific R gene with an inducible C1 gene (or vise versa) would result in tissue specific inducible anthocyanin expression. Tightly controlled expression can also be achieved by expressing R and C1 alleles using promoters with overlapping expression patterns. For example if an R gene driven by a promoter expressing in embryos and tassels was combined with a C1 gene driven by a promoter driving expression to both roots and embryos the resulting transgenic plant would only express anthocyanin in the embryos.

The expression of these regulatory genes is complex. For example, more than 50 naturally occurring alleles of R that condition unique patterns of pigmentation have been described. One allele, R-nj, has been cloned by tagging with the transposable element Ac. R-nj is approximately 90% homologous with the R genes P, S, Lc and B and has been used to isolate an Lc cDNA clone. The protein encoded by the Lc cDNA has been shown to have features characteristic of a transcriptional activator.

In genotypes recessive for two of the above necessary anthocyanin pathway genes, the two anthocyanin genes could be introduced on two separate expression cassettes to identify transformants. In such a combination, two of the above anthocyanin genes could be operably linked to two distinct promoters whose expression patterns are known to overlap temporally, spatially, and/or in a tissue specific manner. In this way, anthocyanin production, through the complementary action of the two transgenes is tightly regulated (for example, reducing the unwanted impact of leaky expression of a single gene). In one embodiment, the method could utilize C1 and R driven by separate promoters whose expression overlaps, for example, the globulin-1 promoter driving C1 and the LEC1 promoter driving R. This would limit anthocyanin production to the embryo. In another embodiment, C1 can be driven by the LEC1 promoter and the R gene is placed behind an inducible promoter such as ln2-2, limiting anthocyanin production to embryos but only in the presence of safener. Many such useful combinations of anthocyanin genes and promoters can be envisaged to represent “complementary pairs” potentially useful for identifying primary transformants and for potential use as markers used in the field and lab to monitor the presence of a transgenic locus in crop germplasm.

Accordingly, a clone encoding for one of these genes can be operably linked to appropriate expression sequences to provide an expression cassette which can be introduced into plant cells by any desired transformation method, such as microprojectile bombardment or Agrobacterium-mediated transformation. Red cells accumulating anthocyanin can be readily detected in transformed cells. Since the accumulation of anthocyanin can be followed in living tissue, expression cassettes comprising an anthocyanin polynucleotide provide a useful reporter/marker gene and transformation vector for maize and other plant cells.

Mutants such as those described in Sainz M B, Goff S A, Chandler V L., Mol Cell Biol., 1997 January;17(1):115-22 may be used. Extensive mutagenesis of a transcriptional activation domain identifies single hydrophobic and acidic amino acids important for activation in vivo.

As noted above, C1 is a transcriptional activator of genes encoding biosynthetic enzymes of the maize anthocyanin pigment pathway. C1 has an amino terminus homologous to Myb DNA-binding domains and an acidic carboxyl terminus that is a transcriptional activation domain in maize and yeast cells. To identify amino acids critical for transcriptional activation, an extensive random mutagenesis of the C1 carboxyl terminus was done. The C1 activation domain is remarkably tolerant of amino acid substitutions, as changes at 34 residues had little or no effect on transcriptional activity. These changes include introduction of helix-incompatible amino acids throughout the C1 activation domain and alteration of most single acidic amino acids, suggesting that a previously postulated amphipathic alpha-helix is not required for activation. Substitutions at two positions revealed amino acids important for transcriptional activation. Replacement of leucine 253 with a proline or glutamine resulted in approximately 10% of wild-type transcriptional activation. Leucine 253 is in a region of C1 in which several hydrophobic residues align with residues important for transcriptional activation by the herpes simplex virus VP16 protein. However, changes at all other hydrophobic residues in C1 indicate that none are critical for C1 transcriptional activation. The other important amino acid in C1 is aspartate 262, as a change to valine resulted in only 24% of wild-type transcriptional activation. Comparison of our C1 results with those from VP16 reveal substantial differences in which amino acids are required for transcriptional activation in vivo by these two acidic activation domains.

Growth stimulation polynucleotides that give cells a growth advantage on medium without growth inhibiting chemicals include but are not limited to Lec1 discussed below, Lec2 (Stone-Sandra-L; Kwong-Linda-W; Yee-Kelly-Matsudaira; Pelletier-Julie; Lepiniec-Loic; Fischer-Robert-L; Goldberg-Robert-B; Harada-John-J. Proceedings-of-the-National-Academy-of-Sciences-of-the-United-States-of-America. Sep. 25, 2001; 98 (20): 11806-11811), AGL15 (U.S. Pat. No. 6,133,435. Down regulation of Pickle (: Ogas,-J.; Kaufmann,-S.; Henderson,-J.; Somerville,-C. Proc Natl Acad Sci USA.,. Nov. 23,1999. v. 96 (24) p. 13839-13844). SERK (V Hecht, J P VielleCalzada, M V Hartog, E D L Schmidt, K Boutilier, U Grossniklaus, S C deVries, Plant Physiology, 2001, Vol 127, Iss 3, pp 803-816), Baby boom (Kim Boutilier: 3rd EPEN meeting Monday Nov. 29 to Tuesday Nov. 30 1999); Knotted-1 (Sinha-Neelima-R; Williams-Rosalind-E; Hake-Sarah, Genes-and-Development., 1993; 7 (5) 787-795); Det2 (Hu, Y., Bao, F., and Li, J., Plant J., 2000 December;24(5):693-701); IPT's (Takei, K., Sakakibara, H., and Sugiyama, T., J Biol Chem., 2001 July 13;276(28):26405-10; Clavata (Clark-Steven-E; Jacobsen-Steven-E; Levin-Joshua-Z; Meyerowitz-Elliot-M, Development, 1996; 122 (5) 1567-1575); Wuschel (Mayer-Klaus-F-X; Schoof-Heiko; Haecker-Achim; Lenhard-Michael; Juergens-Gerd; Laux-Thomas, Cell, Dec. 11, 1998; 95 (6) 805-815); PSK's (Heping Yang, Yoshikatsu Matsubayashi, Kenzo Nakamura, and Youji Sakagami, Plant Physiol, November 2001, Vol. 127, pp. 842-851); Amidohydrolases (Bartel-Bonnie; Fink-Gerald-R, Science, 1995; 268 (5218) 1748-1748); beta-glucosidase's (Brzobohaty,-B.; Moore,-I.; Kristofferson,-P.; Bako,-L.; Campos,-N.; Schell,-J.; Palme,-K., Science,. Nov. 12, 1993, v. 262 (5136) p. 1051-1054); TATA box binding proteins (Li YF, Dubois F, Zhou DX., FEBS Lett., 2001 Feburary 2;489(2-3):187-91); citrate synthase (Koyama H, Kawamura A, Kihara T, Hara T, Takita E, Shibata D., Plant Cell Physiol., 2000 September; 41(9):1030-7); Cell cycle genes (for review see Meijer, M., and Murray, J. A H., Current Opinion in plant Biology, 2001,4:44; -9) such as cyclin B (Doerner,-P.; Jorgensen,-J. E.; You,-R.; Steppuhn,-J.; Lamb,-C., Nature, Apr. 11, 1996. v. 380 (6574) p. 520-523); cyclin D (WO 00/17364) and Cyclin E (U.S. Ser. No. 09/496,444); Down regulation of CKI's (PCT US 01/44038); Cyclin dependent kinases, down regulation of Wee1 (WO 00/37645), E2F; and plant homologs to oncogenes such as ras (Frary-Anne; Nesbitt-T-Clint; Frary-Amy; Grandillo-Silvana; van-der-Knaap-Esther; Cong-Bin; Liu-Jiping; Meller-Jaroslaw; Elber-Ron; Alpert-Kevin-B; Tanksley-Steven-D, Science, Jul. 7, 2000; 289 (5476): 85-88); and down-regulation of homologues to prohibitin (WO 00/15818); and the putative Glutamate Carboxypeptidase AMP1 ( Helliwell,C. A., Chin-Atkins, A. N., Wilson, l. W., Chapple, R., Dennis, E. S., and Chaudhury, A., 2001, The Plant Cell 13:2115-2125). As noted above, it may be necessary in some cases to silence expression of the growth stimulation polynucleotide. Examples of these genes include cell cycle inhibitors such as wee1 and CKI's, negative regulators of somatic embryogenesis such as Pickle and homologues of tumor suppressor proteins such as prohibitin.

LEC1 polypeptides are homologous to the HAP3 subunit of the “CCAAT-box binding factor” class of eukaryotic transcriptional activators (Lotan et aL, 1998, Cell 93:1195-1205). This class of proteins, which consist of Hap2/3/4 and 5, form a heteroligomeric transcriptional complex which appears to activate specific gene sets in eukaryotes. Certain members of this family such as Hap2 and Hap5 appear to be ubiquitously expressed, while different Hap3 members are under developmental or environmental regulation. Plant HAP3 polypeptides can be recognized by a high degree of sequence identity to other HAP3 homologs in the “B domain” of the protein. For example, the B domain for the Arabidopsis LEC1, from amino acid residue 28 to residue 117, shares between 55% and 63% identity (75-85% similarity) to other members of the HAP3 family, including maize (HAP3), chicken, lamprey, Xenopus, human, mouse, Emericella nidulens, Schizosaccharomyces pombe, Saccharomyces cerevisiae and Kluuyveromyces lactis (Lotan et al., 1998). Plant Lec1 sequences can be found in WO 98/37184, WO 99/67405, and WO 00/28058.

It may be desirable to “kick start” somatic embryogenesis by transiently expressing the LEC1 polynucleotide product. This can be done by delivering LEC1 5′capped polyadenylated RNA, expression cassettes containing LEC1 DNA, RNA or LEC1 protein. All of these molecules can be delivered using a biolistics particle gun. For example 5′capped polyadenylated LEC1 RNA can easily be made in vitro using Ambion's mMessage mMachine kit. Other methods for introducing proteins using vir genes are described in WO 99/61619.

Genes of interest are reflective of the commercial markets and interests of those involved in the development of the crop. Crops and markets of interest change, and as developing nations open up world markets, new crops and technologies will emerge also. In addition, as our understanding of agronomic traits and characteristics such as yield and heterosis increases, the choice of genes for transformation will change accordingly. General categories of genes of interest include for example, those genes involved in information, such as zinc fingers, those involved in communication, such as kinases, and those involved in housekeeping, such as heat shock proteins. More specific categories of transgenes, for example, include genes encoding agronomic traits, insect resistance, disease resistance, herbicide resistance, sterility, grain characteristics, and commercial products. Genes of interest include, generally, those involved in oil, starch, carbohydrate, or nutrient metabolism as well as those affecting for example kernel size, sucrose loading, and the like. The quality of grain is reflected in traits such as levels and types of oils, saturated and unsaturated, quality and quantity of essential amino acids, and levels of cellulose.

Grain traits such as oil, starch, and protein content can be genetically altered in addition to using traditional breeding methods. Modifications include increasing content of oleic acid, saturated and unsaturated oils, increasing levels of lysine and sulfur, providing essential amino acids, and also modification of starch. Hordothionin protein modifications are described in U.S. Pat. No. 5,990,389 issued Nov. 23, 1999, U.S. Pat. No. 5,885,801 issued Mar. 23,1999, U.S. Pat. No. 5,885,802 issued Mar. 23,1999 and U.S. Pat. No. 5,703,049 issued Dec. 30,1997; herein incorporated by reference. Another example is lysine and/or sulfur rich seed protein encoded by the soybean 2S albumin described in U.S. Pat. No. 5,850,016 issued Dec. 15, 1998, and the chymotrypsin inhibitor from barley, Williamson et al. (1987) Eur. J. Biochem. 165:99-106, the disclosures of which are herein incorporated by reference.

Derivatives of the coding sequences can be made by site-directed mutagenesis to increase the level of preselected amino acids in the encoded polypeptide. For example, the gene encoding the barley high lysine polypeptide (BHL) is derived from barley chymotrypsin inhibitor WO 98/20133 which is incorporated herein by reference. Other proteins include methionine-rich plant proteins such as from corn (Pedersen et al. (1986) J. Biol. Chem. 261:6279; Kirihara et al. (1988) Gene 71:359; both of which are herein incorporated by reference); and rice (Musumura et al. (1989) Plant Mol. Biol. 12:123, herein incorporated by reference). Other genes encode latex, Floury 2, growth factors, seed storage factors, and transcription factors.

Insect resistance genes may encode resistance to pests that have great yield drag such as rootworm, cutworm, European Corn Borer, and the like. Such genes include, for example Bacillus thuringiensis toxic protein genes (U.S. Pat. Nos. 5,366,892; 5,747,450; 5,737,514; 5,723,756; 5,593,881; Geiseretal. (1986) Gene 48:109); lectins (Van Damme et al. (1994) Plant Mol. Biol. 24:825); and the like.

Genes encoding disease resistance traits include detoxification genes, such as against fumonosin (U.S. Pat. No. 5,792,931, issued Aug. 11,1998); avirulence (avr) and disease resistance genes (Jones et al. (1994) Science 266:789; Martin et al. (1993) Science 262:1432; Mindrinos et al. (1994) Cell 78:1089); and the like.

Herbicide resistance traits may include genes coding for resistance to herbicides that act to inhibit the action of acetolactate synthase (ALS), in particular the sulfonylurea-type herbicides (e.g., the acetolactate synthase (ALS) gene containing mutations leading to such resistance, in particular the S4 and/or Hra mutations), genes coding for resistance to herbicides that act to inhibit action of glutamine synthase, such as phosphinothricin or basta (e.g., the bar gene), or other such genes known in the art. The bar gene encodes resistance to the herbicide basta, the nptll gene encodes resistance to the antibiotics kanamycin and geneticin, and the ALS gene encodes resistance to the herbicide chlorsulfuron.

Sterility genes can also be encoded in an expression cassette and provide an alternative to physical detasseling. Examples of genes used in such ways include male tissue-preferred genes and genes with male sterility phenotypes such as QM, described in U.S. Pat. No. 5,583,210. Other genes include kinases and those encoding compounds toxic to either male or female gametophytic development.

Commercial traits can also be encoded on a gene or genes that could increase for example, starch for ethanol production, or provide expression of proteins. Another commercial use of transformed plants is the production of polymers and bioplastics such as described in U.S. Pat. No. 5,602,321 issued Feb. 11, 1997. Genes such as B-Ketothiolase, PHBase (polyhydroxybutyrate synthase) and acetoacetyl-CoA reductase (see Schubert et al. (1988) J. Bacteriol. 170:5837-5847) facilitate expression of polyhyroxyalkanoates (PHAs).

Genes of medicinal and pharmaceutical uses, such as that encoding avidin and vaccines or proteins produced utilizing plants as factories are also contemplated as part of this invention.

Isolated nucleic acids useful in the present invention can be made using (a) standard recombinant methods, (b) synthetic techniques, or combinations thereof. In some embodiments, the polynucleotides of the present invention will be cloned, amplified, or otherwise constructed from a monocot or dicot. In preferred embodiments the monocot is corn, sorghum, barley, wheat, millet, or rice. Preferred dicots include soybeans, sunflower, canola, alfalfa, potato, or cassava.

Functional fragments may be used in the invention and can be obtained using primers that selectively hybridize under stringent conditions. Primers are generally at least 12 bases in length and can be as high as 200 bases, but will generally be from 15 to 75, preferably from 15 to 50. Functional fragments can be identified using a variety of techniques such as restriction analysis, Southern analysis, primer extension analysis, and DNA sequence analysis.

A plurality of polynucleotides that encode for the identical amino acid sequence may be used in the invention. The degeneracy of the genetic code allows for such “silent variations” which can be used, for example, to selectively hybridize and detect allelic variants of polynucleotides of the present invention. Additionally, the present invention includes isolated nucleic acids comprising allelic variants. The term “allele” as used herein refers to a related nucleic acid of the same gene.

Variants of nucleic acids useful in the invention can be obtained, for example, by oligonucleotide-directed mutagenesis, linker-scanning mutagenesis, mutagenesis using the polymerase chain reaction, and the like. See, for example, Ausubel, pages 8.0.3-8.5.9. Also, see generally, McPherson (ed.), DIRECTED MUTAGENESIS: A Practical Approach, (IRL Press, 1991). Thus, the present invention also encompasses DNA molecules comprising nucleotide sequences that have substantial sequence similarity with the inventive sequences.

Variants may contain individual substitutions, deletions or additions to the nucleic acid or polypeptide sequences that alter, add or delete a single amino acid or a small percentage of amino acids in the encoded sequence. A “conservatively modified variant” is where the alteration results in the substitution of an amino acid with a chemically similar amino acid. When the nucleic acid is prepared or altered synthetically, advantage can be taken of known codon preferences of the intended host.

“Shufflents” produced by sequence shuffling of the polynucleotides to obtain a desired characteristic may also be used in the invention. Sequence shuffling is described in PCT publication No. 96/19256. See also, Zhang, J. H., et al., Proc. Natl. Acad. Sci. USA 94:4504-4509 (1997).

It is also possible to use 5′ and/or 3′ UTR regions for modulation of translation of heterologous coding sequences. Positive sequence motifs include translational initiation consensus sequences (Kozak, Nucleic Acids Res. 15:8125 (1987)) and the 7-methylguanosine cap structure (Drummond et al., Nucleic Acids Res. 13:7375 (1985)). Negative elements include stable intramolecular 5′ UTR stem-loop structures (Muesing et al., Cell 48:691 (1987)) and AUG sequences or short open reading frames preceded by an appropriate AUG in the 5′ UTR (Kozak, supra, Rao et al., Mol. and Cell. Biol. 8:284 (1988)).

Further, the polypeptide-encoding segments of the polynucleotides can be modified to alter codon usage. Altered codon usage can be employed to alter translational efficiency. Codon usage in the coding regions of the polynucleotides of the present invention can be analyzed statistically using commercially available software packages such as “Codon Preference” available from the University of Wisconsin Genetics Computer Group (see Devereaux et al., Nucleic Acids Res. 12:387-395 (1984)) or MacVector 4.1 (Eastman Kodak Co., New Haven, Conn.).

For example, the inventive nucleic acids can be optimized for enhanced expression in plants of interest. See, for example, EPA0359472; WO91/16432; Perlak et al. (1991) Proc. Natl. Acad. Sci. USA 88:3324-3328; and Murray et al. (1989) Nucleic Acids Res. 17:477-498. In this manner, the polynucleotides can be synthesized utilizing plant-preferred codons. See, for example, Murray et al. (1989) Nucleic Acids Res. 17:477-498, the disclosure of which is incorporated herein by reference.

Subsequences comprising isolated nucleic acids containing at least 50 contiguous bases of the nucleotide sequences may be used. For example the isolated nucleic acid includes those comprising at least 50, 60, 75, 100, 250, or 500 contiguous nucleotides of the inventive sequences. Subsequences of the isolated nucleic acid can be used to modulate or detect gene expression by introducing into the subsequences compounds which bind, intercalate, cleave and/or crosslink to nucleic acids.

The nucleic acids may conveniently comprise a multi-cloning site comprising one or more endonuclease restriction sites inserted into the nucleic acid to aid in isolation of the polynucleotide. Also, translatable sequences may be, inserted to aid in the isolation of the translated polynucleotide of the present invention. For example, a hexa-histidine marker sequence provides a convenient means to purify the proteins of the present invention.

A polynucleotide useful in the present invention can be attached to a vector, adapter, promoter, transit peptide or linker for cloning and/or expression of a polynucleotide of the present invention. Additional sequences may be added to such cloning and/or expression sequences to optimize their function in cloning and/or expression, to aid in isolation of the polynucleotide, or to improve the introduction of the polynucleotide into a cell. Use of cloning vectors, expression vectors, adapters, and linkers is well known and extensively described in the art. For a description of such nucleic acids see, for example, Stratagene Cloning Systems, Catalogs 1995, 1996, 1997 (La Jolla, Calif.); and, Amersham Life Sciences, Inc, Catalog '97 (Arlington Heights, Ill.).

Isolated nucleic acid compositions, such as RNA, cDNA, genomic DNA, or a hybrid thereof, can be obtained from plant biological sources using any number of cloning methodologies known to those of skill in the art. In some embodiments, oligonucleotide probes that selectively hybridize, under stringent conditions, to the polynucleotides of the present invention are used to identify the desired sequence in a cDNA or genomic DNA library.

Exemplary total RNA and mRNA isolation protocols are described in Plant Molecular Biology: A Laboratory Manual, Clark, Ed., Springer-Verlag, Berlin (1997); and, Current Protocols in Molecular Biology, Ausubel et al., Eds., Greene Publishing and Wiley-lnterscience, New York (1995). Total RNA and mRNA isolation kits are commercially available from vendors such as Stratagene (La Jolla, Calif.), Clonetech (Palo Alto, Calif.), Pharmacia (Piscataway, N..J.), and 5′-3′ (Paoli, Pa.). See also, U.S. Pat. Nos. 5,614,391; and, 5,459,253.

Typical cDNA synthesis protocols are well known to the skilled artisan and are described in such standard references as: Plant Molecular Biology: A Laboratory Manual, Clark, Ed., Springer-Verlag, Berlin (1997); and, Current Protocols in Molecular Biology, Ausubel et al., Eds., Greene Publishing and Wiley-lnterscience, New York (1995). cDNA synthesis kits are available from a variety of commercial vendors such as Stratagene or Pharmacia.

An exemplary method of constructing a greater than 95% pure full-length cDNA library is described by Carninci et al., Genomics, 37:327-336 (1996). Other methods for producing full-length libraries are known in the art. See, e.g., Edery et al., Mol. Cell Biol.15(6):3363-3371 (1995); and PCT Application WO 96/34981.

It is often convenient to normalize a cDNA library to create a library in which each clone is more equally represented. A number of approaches to normalize cDNA libraries are known in the art. Construction of normalized libraries is described in Ko, Nucl. Acids. Res. 18(19):5705-5711 (1990); Patanjali et al., Proc. Natl. Acad. U.S.A. 88:1943-1947 (1991); U.S. Pat. Nos. 5,482,685 and 5,637,685; and Soares et al., Proc. Natl. Acad. Sci. USA 91:9228-9232 (1994).

Subtracted cDNA libraries are another means to increase the proportion of less abundant cDNA species. See, Foote et al. in, Plant Molecular Biology: A Laboratory Manual, Clark, Ed., Springer-Verlag, Berlin (1997); Kho and Zarbl, Technique 3(2):58-63 (1991); Sive and St. John, Nucl. Acids Res. 16(22):10937 (1988); Current Protocols in Molecular Biology, Ausubel et al., Eds., Greene Publishing and Wiley-Interscience, New York (1995); and, Swaroop et al., Nuc. Acids Res. 19(8):1954 (1991). cDNA subtraction kits are commercially available. See, e.g., PCR-Select (Clontech).

To construct genomic libraries, large segments of genomic DNA are generated by random fragmentation. Examples of appropriate molecular biological techniques and instructions are found in Sambrook et al., Molecular Cloning: A Laboratory Manual, 2nd Ed., Cold Spring Harbor Laboratory, Vols. 1-3 (1989), Methods in Enzymology, Vol. 152: Guide to Molecular Cloning Techniques, Berger and Kimmel, Eds., San Diego: Academic Press, Inc. (1987), Current Protocols in Molecular Biology, Ausubel et al., Eds., Greene Publishing and Wiley-lnterscience, New York (1995); Plant Molecular Biology: A Laboratory Manual, Clark, Ed., Springer-Verlag, Berlin (1997). Kits for construction of genomic libraries are also commercially available.

The cDNA or genomic library can be screened using a probe based upon the sequence of a nucleic acid of the present invention such as those disclosed herein. Probes may be used to hybridize with genomic DNA or cDNA sequences to isolate homologous polynucleotides in the same or different plant species. Those of skill in the art will appreciate that various degrees of stringency of hybridization can be employed in the assay; and either the hybridization or the wash medium can be stringent. The degree of stringency can be controlled by temperature, ionic strength, pH and the presence of a partially denaturing solvent such as formamide.

Typically, stringent hybridization conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide.

Exemplary low stringency conditions include hybridization with a buffer solution of 30 to 35% formamide, 1 M NaCl, 1% SDS (sodium dodecyl sulfate) at 37° C., and a wash in 1× to 2× SSC (20× SSC=3.0 M NaCl/0.3 M trisodium citrate) at 50° C. Exemplary moderate stringency conditions include hybridization in 40 to 45% formamide, 1 M NaCl, 1% SDS at 37° C., and a wash in 0.5× to 1× SSC at 55° C. Exemplary high stringency conditions include hybridization in 50% formamide, 1 M NaCl, 1% SDS at 37° C., and a wash in 0.1× SSC at 60° C. Typically the time of hybridization is from 4 to 16 hours.

An extensive guide to the hybridization of nucleic acids is found in Tijssen, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, New York (1993); and Current Protocols in Molecular Biology, Chapter 2, Ausubel et al., Eds., Greene Publishing and Wiley-lnterscience, New York (1995). Often, cDNA libraries will be normalized to increase the representation of relatively rare cDNAs.

Nucleic acids can be amplified from nucleic acid samples using amplification techniques. For instance, polymerase chain reaction (PCR) technology can be used to amplify the sequences of polynucleotides of the present invention and related polynucleotides directly from genomic DNA or cDNA libraries. PCR and other in vitro amplification methods may also be useful, for example, to clone nucleic acid sequences that code for proteins to be expressed, to make nucleic acids to use as probes for detecting the presence of the desired mRNA in samples, for nucleic acid sequencing, or for other purposes.

Examples of techniques useful for in vitro amplification methods are found in Berger, Sambrook, and Ausubel, as well as Mullis et al., U.S. Pat. No. 4,683,202 (1987); and, PCR Protocols A Guide to Methods and Applications, Innis et al., Eds., Academic Press Inc., San Diego, Calif. (1990). Commercially available kits for genomic PCR amplification are known in the art. See, e.g., Advantage-GC Genomic PCR Kit (Clontech). The T4 gene 32 protein (Boehringer Mannheim) can be used to improve yield of long PCR products. PCR-based screening methods have also been described. Wilfinger et al. describe a PCR-based method in which the longest cDNA is identified in the first step so that incomplete clones can be eliminated from study. BioTechniques, 22(3):481-486 (1997).

Nucleic acids can be amplified from a plant nucleic acid library. The nucleic acid library may be a cDNA library, a genomic library, or a library generally constructed from nuclear transcripts at any stage of intron processing. Libraries can be made from a variety of plant tissues. Good results have been obtained using mitotically active tissues such as-shoot meristems, shoot meristem cultures, embryos, callus and suspension cultures, immature ears and tassels, and young seedlings. The cDNAs of the present invention were obtained from immature zygotic embryo and regenerating callus libraries.

Alternatively, the sequences can be used to isolate corresponding sequences in other organisms, particularly other plants, more particularly, other monocots. In this manner, methods such as PCR, hybridization, and the like can be used to identify such sequences having substantial sequence similarity to the sequences of the invention. See, for example, Sambrook et al. (1989) Molecular Cloning: A Laboratory Manual (2d ed., Cold Spring Harbor Laboratory Press, Plainview, N.Y.). and Innis et al. (1990), PCR Protocols: A Guide to Methods and Applications (Academic Press, New York). Coding sequences isolated based on their sequence identity to the entire inventive coding sequences set forth herein or to fragments thereof are encompassed by the present invention.

The isolated nucleic acids can also be prepared by direct chemical synthesis by methods such as the phosphotriester method of Narang et al., Meth. Enzymol. 68:90-99 (1979); the phosphodiester method of Brown et al., Meth. Enzymol. 68:109-151 (1979); the diethylphosphoramidite method of Beaucage et al., Tetra. Lett. 22:1859-1862 (1981); the solid phase phosphoramidite triester method described by Beaucage and Caruthers, Tetra. Letts. 22(20):1859-1862 (1981), e.g., using an automated synthesizer, e.g., as described in Needham-VanDevanter et al., Nucleic Acids Res. 12:6159-6168 (1984); and, the solid support method of U.S. Pat. No. 4,458,066. Chemical synthesis generally produces a single stranded oligonucleotide. This may be converted into double stranded DNA by hybridization with a complementary sequence, or by polymerization with a DNA polymerase using the single strand as a template. One of skill will recognize that while chemical synthesis of DNA is limited to sequences of about 100 bases, longer sequences may be obtained by the ligation of shorter sequences.

Expression cassettes comprising a visual marker, such as CRC (a fusion of the activation domains of R and C1), and a growth stimulation polynucleotide, such as a Lec1 polynucleotide, are provided. The polynucleotides are operably linked to transcriptional initiation regulatory sequences that will direct the transcription of the polynucleotide in the intended host cell, such as tissues of a transformed plant. The construction of such expression cassettes is well known to those of skill in the art in light of the present disclosure. See, e.g., Sambrook et al.; Molecular Cloning: A Laboratory Manual; Cold Spring Harbor, New York; (1989); Gelvin et al.; Plant Molecular Biology Manual (1990); Plant Biotechnology: Commercial Prospects and Problems, eds. Prakash et al.; Oxford & IBH Publishing Co.; New Delhi, India; (1993); and Heslot et al.; Molecular Biology and Genetic Engineering of Yeasts; CRC Press, Inc., USA; (1992); each incorporated herein in its entirety by reference.

For example, plant expression vectors may include an anthocyanin polynucleotide and a Lec1 polynucleotide under the transcriptional control of a promoter. Such plant expression vectors may also contain, if desired, a promoter regulatory region (e.g., one conferring inducible, constitutive, environmentally- or developmentally-regulated, or cell- or tissue-specific/selective expression), a transcription initiation start site, a ribosome binding site, an RNA processing signal, a transcription termination site, and/or a polyadenylation signal.

Constitutive, tissue-preferred or inducible promoters can be employed. In many cases it is desirable to use promoters that express in callus. Various promoters are suitable such as constitutive promoters, embryo specific promoters, aleurone specific promoters, or cell cycle specific promoters. For visual selection of tissue culture transformants one can drive the visual marker polynucleotide from any promoter as long as it expresses in callus or regenerating callus. A callus preferred promoter allows selection during culture while allowing the marker to be completely off in vegetative plant parts. This strategy allows transformed visually selected plants to be regenerated that have no undesirable visual phenotype. For example CRC could be used for selection in callus and the regenerated plants would be green with no detectable phenotype. The inducible promoter ln2-2 from maize is weakly induced by the auxin in the culture medium and strongly induced by the chemical safener. Co-bombarding Lec1 with ln2-2:CRC into immature embryos will produce red transformed events that can easily be identified in the callusing tissue. When the events are moved to hormone-free regeneration medium (no auxin inducer) the resulting somatic embryos and transformed plants are green. For field screening the inducer safener could be applied to leaves to identify transformants. Plant specific promoters can also be used if plant selection is the goal. These specific examples include only plant components, in fact only maize components, which provides an advantage in obtaining regulatory approval.

Examples of constitutive promoters include the cauliflower mosaic virus (CaMV) 35S transcription initiation region, the 1′- or 2′- promoter derived from T-DNA of Agrobacterium tumefaciens, the nos promoter, the actin promoter, the ubiquitin promoter, the histone H2B promoter (Nakayama et al., 1992, FEBS Lett 30:167-170), the Smas promoter, the cinnamyl alcohol dehydrogenase promoter (U.S. Pat. No. 5,683,439), the Nos promoter, the pEmu promoter, the rubisco promoter, the GRP1-8 promoter, and other transcription initiation regions from various plant genes known in the art.

Examples of inducible promoters are the Adhl promoter which is inducible by hypoxia or cold stress, the Hsp70 promoter which is inducible by heat stress, the PPDK promoter which is inducible by light, the ln2-2 promoter which is safener induced, the ERE promoter which is estrogen induced, Axig1 promoter which is auxin induced and tapetum specific but is also expressed in callus (PCT US 01/22169), and the Pepcarboxylase promoter which is light induced.

Examples of promoters under developmental control include promoters that initiate transcription preferentially in certain tissues, such as leaves, roots, fruit, seeds, or flowers. An exemplary promoter is the anther specific promoter 5126

(U.S. Pat. Nos. 5,689,049 and 5,689,051). Examples of seed-preferred promoters include, but are not limited to, 27 kD gamma zein promoter and waxy promoter, Boronat, A., Martinez, M. C., Reina, M., Puigdomenech, P. and Palau, J.; Isolation and sequencing of a 28 kD glutelin-2 gene from maize: Common elements in the 5′ flanking regions among zein and glutelin genes; Plant Sci. 47:95-102 (1986) and Reina, M., Ponte, I., Guillen, P., Boronat, A. and Palau, J., Sequence analysis of a genomic clone encoding a Zc2 protein from Zea mays W64 A, Nucleic Acids Res. 18(21):6426 (1990) and phaseolin U.S. Pat. No. 5,504,200. See the following site relating to the waxy promoter: Kloesgen, R. B., Gierl,A., Schwarz-Sommer, Z. S. and Saedler, H., Molecular analysis of the waxy locus of Zea mays, Mol. Gen. Genet. 203:237-244 (1986). The DnaJ promoter is found in U.S. Ser. No. 09/387,720. The soybean albumin promoter is found in WO 00/40710. The End1 and End2 promoters express in the endosperm and are found in WO 00/12733. The Jip1, Mi1ps, and Lec1 promoters are found in U.S. Ser. No. 09/718,754. The barley or maize Nuc1 promoter and maize Cim 1 promoter express in nucellus tissue and the maize Ltp2 promoter expresses in the aleurone. These are found in WO 00/11177. The gIb1 and oleosin promoters express in the embryo. The disclosures of each of these are incorporated herein by reference in their entirety.

Either heterologous or non-heterologous (i.e., endogenous) promoters can be employed. These promoters can also be used, for example, in expression cassettes to drive expression of antisense nucleic acids to reduce, increase, or alter concentration and/or composition of proteins in a desired tissue.

If polypeptide expression is desired, it is generally desirable to include a polyadenylation region at the 3′-end of a polynucleotide coding region. The polyadenylation region can be derived from the natural gene, from a variety of other plant genes, or from T-DNA. The 3′ end sequence to be added can be derived from, for example, the nopaline synthase or octopine synthase genes, or alternatively from another plant gene, or less preferably from any other eukaryotic gene.

An intron sequence can be added to the 5′ untranslated region or the coding sequence of the partial coding sequence to increase the amount of the mature message that accumulates. See for example Buchman and Berg, Mol. Cell Biol. 8:4395-4405 (1988); Callis et al., Genes Dev. 1:1183-1200 (1987). Use of maize introns Adh1-S intron 1, 2, and 6, and the Bronze-1 intron are known in the art. See generally, The Maize Handbook, Chapter 116, Freeling and Walbot, Eds., Springer, New York (1994).

Although not required for the invention, the vector or expression cassette may comprise a marker gene which confers a selectable phenotype on plant cells. Usually, the selectable marker gene will encode antibiotic or herbicide resistance. Suitable genes include those coding for resistance to the antibiotics spectinomycin and streptomycin (e.g., the aada gene), the streptomycin phosphotransferase (SPT) gene coding for streptomycin resistance, the neomycin phosphotransferase (NPTII) gene encoding kanamycin or geneticin resistance, the hygromycin phosphotransferase (HPT) gene coding for hygromycin resistance.

Suitable genes coding for resistance to herbicides include those which act to inhibit the action of acetolactate synthase (ALS), in particular the sulfonylurea-type herbicides (e.g., the acetolactate synthase (ALS) gene containing mutations leading to such resistance in particular the S4 and/or Hra mutations), those which act to inhibit action of glutamine synthase, such as phosphinothricin or basta (e.g., the bar gene), or other such genes known in the art. The bar gene encodes resistance to the herbicide basta and the ALS gene encodes resistance to the herbicide chlorsulfuron. While useful in conjunction with the above antibiotic and herbicide-resistance selective markers, a preferred use of LEC1 expression takes advantage of this gene conferring a growth advantage to transformed cells without the need for inhibitory compounds to retard non-transformed growth.

Typical vectors useful for expression of genes in higher plants are well known in the art and include vectors derived from the tumor-inducing (Ti) plasmid of Agrobacterium tumefaciens described by Rogers et al., Meth. In Enzymol. 153:253-277 (1987). Exemplary A. tumefaciens vectors useful herein are plasmids pKYLX6 and pKYLX7 of Schardl et al., Gene, 61:1-11 (1987) and Berger et al., Proc. Natl. Acad. Sci. USA 86:8402-8406 (1989). Another useful vector herein is plasmid pBI101.2 that is available from Clontech Laboratories, Inc. (Palo Alto, Calif.).

A variety of plant viruses that can be employed as vectors are known in the art and include cauliflower mosaic virus (CaMV), geminivirus, brome mosaic virus, and tobacco mosaic virus.

A polynucleotide of interest can be expressed in either sense or anti-sense orientation as desired. In plant cells, it has been shown that antisense RNA inhibits gene expression by preventing the accumulation of mRNA which encodes the enzyme of interest, see, e.g., Sheehy et al., Proc. Natl. Acad. Sci. USA 85:8805-8809 (1988); and Hiatt et al., U.S. Pat. No. 4,801,340.

Another method of suppression is sense suppression. Introduction of nucleic acid configured in the sense orientation has been shown to be an effective means by which to block the transcription of target genes. For an example of the use of this method to modulate expression of endogenous genes see, Napoli et al., The Plant Cell 2:279-289 (1990) and U.S. Pat. No. 5,034,323.

Recent work has shown suppression with the use of double stranded RNA. Such work is described in Tabara et al., Science 282:5388:430-431 (1998). Catalytic RNA molecules or ribozymes can also be used to inhibit expression of plant genes. The inclusion of ribozyme sequences within antisense RNAs confers RNA-cleaving activity upon them, thereby increasing the activity of the constructs. The design and use of target RNA-specific ribozymes is described in Haseloff et al., Nature 334:585-591 (1988).

A variety of cross-linking agents, alkylating agents and radical generating species as pendant groups on polynucleotides of the present invention can be used to bind, label, detect, and/or cleave nucleic acids. For example, Vlassov, V. V., et al., Nucleic Acids Res (1986) 14:4065-4076, describe covalent bonding of a single-stranded DNA fragment with alkylating derivatives of nucleotides complementary to target sequences. A report of similar work by the same group is that by Knorre, D. G., et al., Biochimie (1985) 67:785-789. Iverson and Dervan also showed sequence-specific cleavage of single-stranded DNA mediated by incorporation of a modified nucleotide which was capable of activating cleavage (J. Am. Chem. Soc. (1987) 109:1241-1243). Meyer, R. B., et al., J. Am. Chem. Soc. (1989) 111:8517-8519, effect covalent crosslinking to a target nucleotide using an alkylating agent complementary to the single-stranded target nucleotide sequence. A photoactivated crosslinking to single-stranded oligonucleotides mediated by psoralen was disclosed by Lee, B. L., et al., Biochemistry (1988) 27:3197-3203. Use of crosslinking in triple-helix forming probes was also disclosed by Home, et al., J. Am. Chem. Soc. (1990) 112:2435-2437. Use of N4, N4-ethanocytosine as an alkylating agent to crosslink to single-stranded oligonucleotides has also been described by Webb and Matteucci, J. Am. Chem. Soc. (1986) 108:2764-2765; Nucleic Acids Res (1986) 14:7661-7674; Feteritz et al., J. Am. Chem. Soc. 113:4000 (1991). Various compounds to bind, detect, label, and/or cleave nucleic acids are known in the art. See, for example, U.S. Pat. Nos. 5,543,507; 5,672,593; 5,484,908; 5,256,648; and, 5,681,941.

Proteins useful in the present invention include proteins derived from the native protein by deletion (so-called truncation), addition or substitution of one or more amino acids at one or more sites in the native protein. Such variants may result from, for example, genetic polymorphism or from human manipulation. Methods for such manipulations are generally known in the art.

For example, amino acid sequence variants of the polypeptide can be prepared by mutations in the cloned DNA sequence encoding the native protein of interest. Methods for mutagenesis and nucleotide sequence alterations are well known in the art. See, for example, Walker and Gaastra, eds. (1983) Techniques in Molecular Biology (MacMillan Publishing Company, New York); Kunkel (1985) Proc. Natl. Acad. Sci. USA 82:488-492; Kunkel et al. (1987) Methods Enzymol. 154:367-382; Sambrook et al. (1989) Molecular Cloning: A Laboratory Manual (Cold Spring Harbor, New York); U.S. Pat. No. 4,873,192; and the references cited therein; herein incorporated by reference. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be preferred.

In constructing variants of the proteins of interest, modifications to the nucleotide sequences encoding the variants will be made such that variants possess the desired activity.

The isolated proteins useful in the present invention include a polypeptide comprising at least 50 contiguous amino acids encoded by a polynucleotide of interest, or polypeptides which are conservatively modified variants thereof. The proteins of the present invention or variants thereof can comprise any number of contiguous amino acid residues from a polypeptide of interest, wherein that number is selected from the group of integers consisting of from 50 to the number of residues in a full-length polypeptide. Optionally, this subsequence of contiguous amino acids is at least 50, 60, 70, 80, 90, 100, 200, 300 amino acids in length or more.

The present invention includes catalytically active polypeptides (i.e., enzymes). Catalytically active polypeptides will generally have a specific activity of at least 20%, 30%, 40%, 50%, 60%, 70%, 80%, 90%, 95%, or 100% that of the native (non-synthetic), endogenous polypeptide. Further, the substrate specificity (k_{cat}/Kₘ) is optionally substantially similar to the native (non-synthetic), endogenous polypeptide. Typically, the Kₘ will be at least 30%, 40%, 50%, 60%, 70%, 80%, 90%, 95%, or 100% that of the native (non-synthetic), endogenous polypeptide. Methods of assaying and quantifying measures of enzymatic activity and substrate specificity (k_{cat}/Kₘ), are well known to those of skill in the art.

The present invention includes modifications that can be made to an inventive protein. In particular, it may be desirable to diminish the activity of the LEC1 or anthocyanin gene. Other modifications may be made to facilitate the cloning, expression, or incorporation of the targeting molecule into a fusion protein. Such modifications are well known to those of skill in the art and include, for example, a methionine added at the amino terminus to provide an initiation site, or additional amino acids (e.g., poly His) placed on either terminus to create conveniently located restriction sites or termination codons or purification sequences.

A typical host cell includes bacteria, yeast, insect, mammalian, or plant cells. The cells produce the protein in a non-natural condition (e.g., in quantity, composition, location, and/or time), because they have been genetically altered through human intervention to do so. Typically, an intermediate host cell will be used in the practice of this invention to increase the copy number of the expression cassette. With an increased copy number, the expression cassette containing the gene of interest can be isolated in significant quantities for introduction into the desired plant cells.

Host cells that can be used in the practice of this invention include prokaryotes, including bacterial hosts such as Eschericia coli, Salmonella typhimurium, and Serratia marcescens. Eukaryotic hosts such as yeast or filamentous fungi may also be used in this invention. Since these hosts are also microorganisms, it will be essential to ensure that plant promoters which do not cause expression of the polypeptide in bacteria are used in the vector.

Commonly used prokaryotic control sequences include such commonly used promoters as the beta lactamase (penicillinase) and lactose (lac) promoter systems (Chang et al., Nature 198:1056 (1977)), the tryptophan (trp) promoter system (Goeddel et al., Nucleic Acids Res. 8:4057 (1980)) and the lambda derived P L promoter and N-gene ribosome binding site (Shimatake et al., Nature 292:128 (1981)). The inclusion of selection markers in DNA vectors transfected in E. coli is also useful. Examples of such markers include genes specifying resistance to ampicillin, tetracycline, or chloramphenicol.

The vector is selected to allow introduction into the appropriate host cell. Bacterial vectors are typically of plasmid or phage origin. Expression systems for expressing a protein of the present invention are available using Bacillus sp. and Salmonella (Palva et al., Gene 22:229-235 (1983); Mosbach et al., Nature 302:543-545 (1983)).

Synthesis of heterologous proteins in yeast is well known. See Sherman, F., et al., Methods in Yeast Genetics, Cold Spring Harbor Laboratory (1982). Two widely utilized yeast for production of eukaryotic proteins are Saccharomyces cerevisiae and Pichia pastoris. Vectors, strains, and protocols for expression in Saccharomyces and Pichia are known in the art and available from commercial suppliers (e.g., Invitrogen). Suitable vectors usually have expression control sequences, such as promoters, including 3-phosphoglycerate kinase or alcohol oxidase, and an origin of replication, termination sequences and the like as desired. A protein, once expressed, can be isolated from yeast by lysing the cells and applying standard protein isolation techniques to the lysates. The monitoring of the purification process can be accomplished by using Western blot techniques or radioimmunoassay of other standard immunoassay techniques.

Proteins can also be constructed using non-cellular synthetic methods. Solid phase synthesis of proteins of less than about 50 amino acids in length may be accomplished by attaching the C-terminal amino acid of the sequence to an insoluble support followed by sequential addition of the remaining amino acids in the sequence. Techniques for solid phase synthesis are described by Barany and Merrifield, Solid-Phase Peptide Synthesis, pp. 3-284 in The Peptides: Analysis, Synthesis, Biology. Vol. 2: Special Methods in Peptide Synthesis, Part A.; Merrifield et al., J. Am. Chem. Soc. 85:2149-2156 (1963), and Stewart et al., Solid Phase Peptide Synthesis, 2nd ed., Pierce Chem. Co., Rockford, III. (1984). Proteins of greater length may be synthesized by condensation of the amino and carboxy termini of shorter fragments. Methods of forming peptide bonds by activation of a carboxy terminal end (e.g., by the use of the coupling reagent N,N′-dicycylohexylcarbodiimide)) is known to those of skill.

The proteins, recombinant or synthetic, may be purified to substantial purity by standard techniques well known in the art, including detergent solubilization, selective precipitation with such substances as ammonium sulfate, column chromatography, immunopurification methods, and others. See, for instance, R. Scopes, Protein Purification: Principles and Practice, Springer-Verlag: New York (1982); Deutscher, Guide to Protein Purification, Academic Press (1990). For example, antibodies may be raised to the proteins as described herein. Purification from E. coli can be achieved following procedures described in U.S. Pat. No. 4,511,503. Detection of the expressed protein is achieved by methods known in the art and include, for example, radioimmunoassays, Western blotting techniques or immunoprecipitation.

In some embodiments, the content and/or composition of polypeptides in a plant may be modulated by altering, in vivo or in vitro, the promoter of a non-isolated gene of the present invention to up- or down-regulate gene expression. In some embodiments, the coding regions of native genes of the present invention can be altered via substitution, addition, insertion, or deletion to decrease activity of the encoded enzyme. See, e.g., Kmiec, U.S. Pat. No. 5,565,350; Zarling et al., PCT/US93/03868. One method of down-regulation of the protein involves using PEST sequences that provide a target for degradation of the protein.

In some embodiments, an isolated nucleic acid (e.g., a vector) comprising a promoter sequence is transfected into a plant cell. Subsequently, a plant cell comprising the promoter operably linked to a polynucleotide of the present invention is selected for by means known to those of skill in the art such as Southern blot, DNA sequencing, or PCR analysis using primers specific to the promoter and to the gene and detecting amplicons produced therefrom. A plant or plant part altered or modified by the foregoing embodiments is grown under plant forming conditions for a time sufficient to modulate the concentration and/or composition of polypeptides in the plant. Plant forming conditions are well known in the art.

In general, concentration or composition is increased or decreased by at least 5%, 10%, 20%, 30%, 40%, 50%, 60%, 70%, 80%, 90%, or 95% relative to a native control plant, plant part, or cell lacking the aforementioned expression cassette. Modulation in the present invention may occur during and/or subsequent to growth of the plant to the desired stage of development. Modulating nucleic acid expression temporally and/or in particular tissues can be controlled by employing the appropriate promoter operably linked to a polynucleotide of the present invention in, for example, sense or antisense orientation as discussed in greater detail, supra. Induction of expression of a polynucleotide of the present invention can also be controlled by exogenous administration of an effective amount of inducing compound. Inducible promoters and inducing compounds which activate expression from these promoters are well known in the art. The polypeptides of the present invention can be modulated in monocots or dicots, preferably maize, soybeans, sunflower, sorghum, canola, wheat, alfalfa, rice, barley and millet.

Means of detecting the proteins of the present invention are not critical aspects of the present invention. The proteins can be detected and/or quantified using any of a number of well-recognized immunological binding assays (see, e.g., U.S. Pat. Nos. 4,366,241; 4,376,110; 4,517,288; and 4,837,168). For a review of the general immunoassays, see also Methods in Cell Biology, Vol. 37: Antibodies in Cell Biology, Asai, Ed., Academic Press, Inc. New York (1993); Basic and Clinical Immunology 7th Edition, Stites & Terr, Eds. (1991). Moreover, the immunoassays of the present invention can be performed in any of several configurations, e.g., those reviewed in Enzyme Immunoassay, Maggio, Ed., CRC Press, Boca Raton, Fla. (1980); Tijan, Practice and Theory of Enzyme Immunoassays, Laboratory Techniques in Biochemistry and Molecular Biology, Elsevier Science Publishers B.V., Amsterdam (1985); Harlow and Lane, supra; Immunoassay: A Practical Guide, Chan, Ed., Academic Press, Orlando, Fla. (1987); Principles and Practice of Immunoassays, Price and Newman Eds., Stockton Press, NY (1991); and Non-isotopic Immunoassays, Ngo, Ed., Plenum Press, NY (1988).

Typical methods include Western blot (immunoblot) analysis, analytic biochemical methods such as electrophoresis, capillary electrophoresis, high performance liquid chromatography (HPLC), thin layer chromatography (TLC), hyperdiffusion chromatography, and the like, and various immunological methods such as fluid or gel precipitin reactions, immunodiffusion (single or double), immunoelectrophoresis, radioimmunoassays (RIAs), enzyme-linked immunosorbent assays (ELISAs), immunofluorescent assays, and the like.

Non-radioactive labels are often attached by indirect means. Generally, a ligand molecule (e.g., biotin) is covalently bound to the molecule. The ligand then binds to an anti-ligand (e.g., streptavidin) molecule which is either inherently detectable or covalently bound to a signal system, such as a detectable enzyme, a fluorescent compound, or a chemiluminescent compound. A number of ligands and anti-ligands can be used. Where a ligand has a natural anti-ligand, for example, biotin, thyroxine, and cortisol, it can be used in conjunction with the labeled, naturally occurring anti-ligands. Alternatively, any haptenic or antigenic compound can be used in combination with an antibody.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidoreductases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems which may be used, see, U.S. Pat. No. 4,391,904, which is incorporated herein by reference.

Some assay formats do not require the use of labeled components. For instance, agglutination assays can be used to detect the presence of the target antibodies. In this case, antigen-coated particles are agglutinated by samples comprising the target antibodies. In this format, none of the components need be labeled and the presence of the target antibody is detected by simple visual inspection.

The proteins can be used for identifying compounds that bind to (e.g., substrates), and/or increase or decrease (i.e., modulate) the enzymatic activity of, catalytically active polypeptides of the present invention. The method comprises contacting a polypeptide of the present invention with a compound whose ability to bind to or modulate enzyme activity is to be determined. The polypeptide employed will have at least 20%, 30%, 40%, 50%, 60%, 70%, 80%, 90%, or 95% of the specific activity of the native, full-length polypeptide of the present invention (e.g., enzyme). Methods of measuring enzyme kinetics are well known in the art. See, e.g., Segel, Biochemical Calculations, 2^{nd} ed., John Wiley and Sons, New York (1976).

Antibodies can be raised to a protein, including individual, allelic, strain, or species variants, and fragments thereof, both in their naturally occurring (full-length) forms and in recombinant forms. Additionally, antibodies are raised to these proteins in either their native configurations or in non-native configurations. Anti-idiotypic antibodies can also be generated. Many methods of making antibodies are. known to persons of skill.

In some instances, it is desirable to prepare monoclonal antibodies from various mammalian hosts, such as mice, rodents, primates, humans, etc. Description of techniques for preparing such monoclonal antibodies are found in, e.g., Basic and Clinical Immunology, 4th ed., Stites et al., Eds., Lange Medical Publications, Los Altos, Calif., and references cited therein; Harlow and Lane, Supra; Goding, Monoclonal Antibodies: Principles and Practice, 2nd ed., Academic Press, New York, NY (1986); and Kohler and Milstein, Nature 256:495-497 (1975).

Other suitable techniques involve selection of libraries of recombinant antibodies in phage or similar vectors (see, e.g., Huse et al., Science 246:1275-1281 (1989); and Ward et al., Nature 341:544-546 (1989); and Vaughan et al., Nature Biotechnology, 14:309-314 (1996)). Alternatively, high avidity human monoclonal antibodies can be obtained from transgenic mice comprising fragments of the unrearranged human heavy and light chain Ig loci (i.e., minilocus transgenic mice). Fishwild et al., Nature Biotech., 14:845-851 (1996). Also, recombinant immunoglobulins may be produced. See, Cabilly, U.S. Pat. No. 4,816,567; and Queen et al., Proc. Natl. Acad. Sci. 86:10029-10033 (1989).

The antibodies can be used for affinity chromatography in isolating proteins of the present invention, for screening expression libraries for particular expression products such as normal or abnormal protein or for raising anti-idiotypic antibodies which are useful for detecting or diagnosing various pathological conditions related to the presence of the respective antigens.

Frequently, the proteins and antibodies will be labeled by joining, either covalently or non-covalently, a substance which provides for a detectable signal. A wide variety of labels and conjugation techniques are known and are reported extensively in both the scientific and patent literature. Suitable labels include radionucleotides, enzymes, substrates, cofactors, inhibitors, fluorescent moieties, chemiluminescent moieties, magnetic particles, and the like.

The method of transformation/transfection is not critical to the invention; various methods of transformation or transfection are currently available. As newer methods are available to transform crops or other host cells they may be directly applied. Accordingly, a wide variety of methods have been developed to insert a DNA sequence into the genome of a host cell to obtain the transcription and/or translation of the sequence to effect phenotypic changes in the organism. Thus, any method which provides for efficient transformation/transfection may be employed.

A DNA sequence coding for the desired polynucleotide of the present invention, for example a cDNA or a genomic sequence encoding a full-length protein, can be used to construct an expression cassette which can be introduced into the desired plant. Isolated nucleic acid acids of the present invention can be introduced into plants according techniques known in the art. Generally, expression cassettes as described above and suitable for transformation of plant cells are prepared.

Techniques for transforming a wide variety of higher plant species are well known and described in the technical, scientific, and patent literature. See, for example, Weising et al., Ann. Rev. Genet. 22:421-477 (1988). For example, the DNA construct may be introduced directly into the genomic DNA of the plant cell using techniques such as particle bombardment, electroporation, PEG poration, silicon fiber delivery, or microinjection of plant cell protoplasts or embryogenic callus. See, e.g., Tomes et al., Direct DNA Transfer into Intact Plant Cells Via Microprojectile Bombardment. pp. 197-213 in Plant Cell, Tissue and Organ Culture, Fundamental Methods. eds. O. L. Gamborg and G. C. Phillips. Springer-Verlag Berlin Heidelberg New York, 1995. Alternatively, the DNA constructs may be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria. See, U.S. Pat. No. 5,591,616.

The introduction of DNA constructs using polyethylene glycol precipitation is described in Paszkowski et al., Embo J. 3:2717-2722 (1984). Electroporation techniques are described in From et al., Proc. Natl. Acad. Sci. 82:5824 (1985). Ballistic transformation techniques are described in Klein et al., Nature 327:70-73 (1987).

Agrobacterium tumefaciens-meditated transformation techniques are well described in the scientific literature. See, for example Horsch et al., Science 233:496-498 (1984), and Fraley et al., Proc. Natl. Acad. Sci. 80:4803 (1983). For instance, Agrobacterium transformation of maize is described in WO 98/32326. Agrobacterium transformation of soybean is described in U.S. Pat. No. 5,563,055.

Other methods of transfection or transformation include (1) Agrobacterium rhizogenes-mediated transformation (see, e.g., Lichtenstein and Fuller In: Genetic Engineering, Vol. 6, PWJ Rigby, Ed., London, Academic Press, 1987; and Lichtenstein, C. P., and Draper, J,. In: DNA Cloning, Vol. 11, D. M. Glover, Ed., Oxford, IRI Press, 1985), Application PCT/US87/02512 (WO 88/02405 published Apr. 7, 1988) describes the use of A. rhizogenes strain A4 and its Ri plasmid along with A. tumefaciens vectors pARC8 or pARC16 (2) liposome-mediated DNA uptake (see, e.g., Freeman et al., Plant Cell Physiol. 25:1353, (1984)), (3) the vortexing method (see, e.g., Kindle, Proc. Natl. Acad. Sci. USA 87:1228, (1990)).

DNA can also be introduced into plants by direct DNA transfer into pollen as described by Zhou et al., Methods in Enzymology, 101:433 (1983); D. Hess, Intern Rev. Cytol., 107:367 (1987); Luo et al., Plane Mol. Biol. Reporter, 6:165 (1988). Expression of polypeptide coding polynucleotides can be obtained by injection of the DNA into reproductive organs of a plant as described by Pena et al., Nature, 325:274 (1987). DNA can also be injected directly into the cells of immature embryos and the rehydration of desiccated embryos as described by Neuhaus et aL, Theor. Appi. Genet., 75:30 (1987); and Benbrook et al., in Proceedings Bio Expo 1986, Butterworth, Stoneham, Mass., pp. 27-54 (1986).

Animal and lower eukaryotic (e.g., yeast) host cells are competent or rendered competent for transfection by various means. There are several well-known methods of introducing DNA into animal cells. These include: calcium phosphate precipitation, fusion of the recipient cells with bacterial protoplasts containing the DNA, treatment of the recipient cells with liposomes containing the DNA, DEAE dextran, electroporation, biolistics, and micro-injection of the DNA directly into the cells. The transfected cells are cultured by means well known in the art. Kuchler, R. J., Biochemical Methods in Cell Culture and Virology, Dowden, Hutchinson and Ross, Inc. (1977).

Using the following methods for controlling cell division, it is possible to alter plant tissue culture media components to suppress callus growth in a plant species of interest (culture media contains multiple components that potentially could be adjusted to impart this effect). Such conditions would not impart a negative or toxic in vitro environment for wild-type tissue, but instead would simply reduce callus growth. Introducing a transgene such as LEC1 will stimulate somatic embryogenesis and/or growth in the transformed cells or tissue, providing a clear differential growth screen useful for identifying transformants.

Altering a wide variety of media components can modulate somatic embryogenesis and growth rate (either stimulating or suppressing embryogenesis depending on the species and particular media component). Examples of media components which, when altered, can stimulate or suppress growth include; 1) the basal medium itself (macronutrient, micronutrients and vitamins; see T. A. Thorpe, 1981 for review, “Plant Tissue Culture: Methods and Applications in Agriculture”, Academic Press, NY), 2) plant phytohormones such as auxins (indole acetic acid, indole butyric acid, 2,4-dichlorophenoxyacetic acid, naphthaleneacetic acid, picloram, dicamba and other functional analogues), cytokinins (zeatin, kinetin, benzyl amino purine, 2-isopentyl adenine and functionally-related compounds) abscisic acid, adenine, and gibberellic acid, 3) and other compounds that exert “growth regulator” effects such as coconut water, casein hydrolysate, and proline, and 4) the type and concentration of gelling agent, pH and sucrose concentration. 5) non-lethal concentrations of antibiotics or herbicides

Changes in the individual components listed above (or in some cases, combinations of components) have been demonstrated in the literature to modulate in vitro somatic embryogenesis across a wide range of dicotyledonous and monocotyledonous species. For a compilation of examples, see E. F. George et al., 1987. Plant Tissue Culture Media. Vol.1: Formulations and Uses. Exergetics, Ltd., Publ., Edington, England.

Transformed plant cells which are derived by any of the above transformation techniques can be cultured to regenerate a whole plant which possesses the transformed genotype. Such regeneration techniques often rely on manipulation of certain phytohormones in a tissue culture growth medium, typically relying on a biocide and/or herbicide marker that has been introduced together with a polynucleotide of the present invention. For transformation and regeneration of maize see, Gordon-Kamm et al., The Plant Cell, 2:603-618 (1990).

Plants cells transformed with a plant expression vector can be regenerated, e.g., from single cells, callus tissue or leaf discs according to standard plant tissue culture techniques. It is well known in the art that various cells, tissues, and organs from almost any plant can be successfully cultured to regenerate an entire plant. Plant regeneration from cultured protoplasts is described in Evans et al., Protoplasts Isolation and Culture, Handbook of Plant Cell Culture, Macmillan Publishing Company, New York, pp. 124-176 (1983); and Binding, Regeneration of Plants, Plant Protoplasts, CRC Press, Boca Raton, pp. 21-73 (1985).

The regeneration of plants containing the foreign gene introduced by Agrobacterium can be achieved as described by Horsch et al., Science, 227:1229-1231 (1985) and Fraley et al., Proc. Natl. Acad. Sci. U.S.A. 80:4803 (1983). This procedure typically produces shoots within two to four weeks and these transformant shoots are then transferred to an appropriate root-inducing medium containing the selective agent and an antibiotic to prevent bacterial growth. Transgenic plants of the present invention may be fertile or sterile.

Regeneration can also be obtained from plant callus, explants, organs, or parts thereof. Such regeneration techniques are described generally in Klee et al., Ann. Rev. of Plant Phys. 38:467-486 (1987). The regeneration of plants from either single plant protoplasts or various explants is well known in the art. See, for example, Methods for Plant Molecular Biology, A. Weissbach and H. Weissbach, eds., Academic Press, Inc., San Diego, Calif. (1988). For maize cell culture and regeneration see generally, The Maize Handbook, Freeling and Walbot, Eds., Springer, New York (1994); Com and Corn Improvement, 3^{rd} edition, Sprague and Dudley Eds., American Society of Agronomy, Madison, Wis. (1988).

One of skill will recognize that after the expression cassette is stably incorporated in transgenic plants and confirmed to be operable, it can be introduced into other plants by sexual crossing. Any of a number of standard breeding techniques can be used, depending upon the species to be crossed.

In vegetatively propagated crops, mature transgenic plants can be propagated by the taking of cuttings, via production of apomictic seed, or by tissue culture techniques to produce multiple identical plants. Selection of desirable transgenics is made and new varieties are obtained and propagated vegetatively for commercial use. In seed propagated crops, mature transgenic plants can be self-crossed to produce a homozygous inbred plant. The inbred plant produces seed containing the newly introduced heterologous nucleic acid. These seeds can be grown to produce plants that would produce the selected phenotype.

Parts obtained from the regenerated plant, such as flowers, seeds, leaves, branches, fruit, and the like are included in the invention, provided that these parts comprise cells comprising the isolated nucleic acid of the present invention. Progeny and variants, and mutants of the regenerated plants are also included within the scope of the invention, provided that these parts comprise the introduced nucleic acid sequences.

Transgenic plants expressing a selectable marker or visual marker can be screened for transmission of the nucleic acid of the present invention by, for example, standard immunoblot and DNA detection techniques or visually by expression of the anthocyanin polypeptides. Transgenic lines are also typically evaluated on levels of expression of the heterologous nucleic acid. Expression at the RNA level can be determined initially to identify and quantitate expression-positive plants. Standard techniques for RNA analysis can be employed and include PCR amplification assays using oligonucleotide primers designed to amplify only the heterologous RNA templates and solution hybridization assays using heterologous nucleic acid-specific probes. The RNA-positive plants can then be analyzed for protein expression by Western immunoblot analysis using the specifically reactive antibodies of the present invention. In addition, in situ hybridization and immunocytochemistry according to standard protocols can be done using heterologous nucleic acid specific polynucleotide probes and antibodies, respectively, to localize sites of expression within transgenic tissue. Generally, a number of transgenic lines are usually screened for the incorporated nucleic acid to identify and select plants with the most appropriate expression profiles.

One embodiment includes a transgenic plant that is homozygous for the added heterologous nucleic acid; i.e., a transgenic plant that contains two added nucleic acid sequences, one gene at the same locus on each chromosome of a chromosome pair. A homozygous transgenic plant can be obtained by sexually mating (selfing) a heterozygous transgenic plant that contains a single added heterologous nucleic acid, germinating some of the seed produced and analyzing the resulting plants produced for altered expression of a polynucleotide of the present invention relative to a control plant (i.e., native, non-transgenic). Back-crossing to a parental plant and out-crossing with a non- transgenic plant are also contemplated. Alternatively, propagation of heterozygous transgenic plants could be accomplished through apomixis.

In some applications it may be desirable to link an agronomic gene of interest to the visual marker, such as the anthocyanin polypeptide. This will allow one to use expression of the visual marker (tissue specific, inducible, or constitutive) to screen for plants containing the agronomic gene of interest. This can be done using a variety of methods the preferred method using the two T-DNA Agrobacterium co-transformation vector (see U.S. Pat. No. 5,981,840). For example, cells could be transformed with a vector containing two T-DNA's, one containing Lec1 to confer a growth advantage, and the other containing an anthocyanin polypeptide with the gene of interest. These two T-DNA's could then be separated from one another in segregating progeny allowing one to identify plants containing the agronomic gene: linked to the visual marker without the growth conferring lecd expression cassette. Alternatively, the lec1 and visual marker polynucleotides could be placed in one T-DNA and the agronomic gene of interest could be placed in the other T-DNA. The progeny without the visual marker could then be screened for the presence of only the agronomic gene. Using these system plants could be obtained that contain only the agronomic gene of interest.

The present invention provides a method of genotyping a plant. Genotyping provides a means of distinguishing homologs of a chromosome pair and can be used to differentiate segregants in a plant population. Molecular marker methods can be used for phylogenetic studies, characterizing genetic relationships among crop varieties, identifying crosses or somatic hybrids, localizing chromosomal segments affecting monogenic traits, map based cloning, and the study of quantitative inheritance. See, e.g., Plant Molecular Biology: A Laboratory Manual, Chapter 7, Clark, Ed., Springer-Verlag, Berlin (1997). For molecular marker methods, see generally, The DNA Revolution by Andrew H. Paterson 1996 (Chapter 2) in: Genome Mapping in Plants (ed. Andrew H. Paterson) by Academic Press/R. G. Landis Company, Austin, Tex., pp.7-21.

The particular method of genotyping may employ any number of molecular marker analytic techniques such as, but not limited to, restriction fragment length polymorphisms (RFLPs). RFLPs are the product of allelic differences between DNA restriction fragments caused by nucleotide sequence variability. Thus, the present invention further provides a means to follow segregation of a gene or nucleic acid of the present invention as well as chromosomal sequences genetically linked to these genes or nucleic acids using such techniques as RFLP analysis.

Plants which can be used in the method of the invention include monocotyledonous and dicotyledonous plants. Preferred plants include maize, wheat, rice, barley, oats, sorghum, millet, rye, soybean, sunflower, alfalfa, canola and cotton. Of these monocots are preferred.

Seeds derived from plants regenerated from transformed plant cells, plant parts or plant tissues, or progeny derived from the regenerated transformed plants, may be used directly as feed or food, or further processing may occur.

All publications cited in this application are herein incorporated by reference to the same extent as if each individual publication or patent application was specifically and individually indicated to be incorporated by reference.

The present invention will be further described by reference to the following detailed examples. It is understood, however, that there are many extensions, variations, and modifications on the basic theme of the present invention beyond that shown in the examples and description, which are within the spirit and scope of the present invention.

Inducible or tissue-specific expression of a visual marker could be used for both transformant identification and field selection of transgenic material. If unlinked to a negative selection marker, controlled pigmentation could be a viable alternative to other non-visual methods for identification of transgenic plants such as herbicide applications and ELISA's. For demonstration purposes the activation domains of C1 are fused to the activation domains of R. The resulting fusion product (CRC) activates the anthocyanin pathway and has the advantage of being easier to work with. In some cases it will be advantageous to use R and C separately rather than the CRC gene fusion. Mutations to C1, which modulate its transactivation of other genes are also advantageous in this invention.

### EXAMPLES

### Example 1

Inducible tissue specific anthocyanin expression

In genotypes without a dominant R or C1 allele, both R and C1 are required for activation of the anthocyanin pathway. By placing one of these under an inducible promoter and the other under a tissue specific promoter it is possible to have inducible expression in a specific tissue type. For example an inducible promoter driving a C1 family member in combination with an embryo specific promoter driving an R family member would result in inducible embryo specific anthocyanin expression. This method for inducible expression would provide a means to identify transformants in culture and in the field that could be turned off for product development. In this prophetic example particle gun transformation is used but a more preferred method would be transformation using an Agrobacterium co-transformation vector (see U.S. Pat. No. 5,981,840). Using the co-transformation system the gene providing the growth advantage (lec1) could be on a separate T-DNA from an agronomic gene linked to anthocyanin polynucleotide(s). Thus, the agronomic gene would be linked to the pigment marker and easily segregated away from the gene providing the growth advantage. In another embodiment the anthocyanin activating genes could be placed in the same T-DNA as the gene providing the growth advantage with the agronomic gene on the other T-DNA. In this case pigmentation and callus growth stimulation could be used for primary transformant selection but eliminated via segregation for product development. In yet another embodiment an isolated lec1 gene may already be present in the cells to be transformed, i.e. the target cells were transformed with lec1 in a previous generation.

Transformations can be performed as follows: Using the genotype High type 11 (Hi-II) as an example, plants can be grown to maturity and immature ears harvested 8-10 days after pollination, ears are surface sterilized in 50% Clorox bleach plus 0.5% Micro detergent for 20 minutes, and rinsed three times with sterile water. The immature embryos are excised and placed embryo axis side down (scutellum side up), 25 embryos per plate, and cultured for 3-5 days on 560P medium, an N6-based medium containing Eriksson's vitamins, thiamine, sucrose, 2,4-D (2 mg/L), and silver nitrate. Two to three hours before bombardment these embryos are transferred to high osmotic 560Y medium,N6-based medium containing Eriksson's vitamins, thiamine, 12% sucrose, 2,4-D (1 mg/L), and silver nitrate. Plasmid DNA is precipitated onto 0.6 μm (average diameter) gold pellets using a CaCl₂ precipitation procedure as follows: 50 μl prepared gold particles in water, 10 μl DNA (used at a concentration of 0.1 μg/μl) in TrisEDTA buffer (1 μg total), 50 μl 2.5 M CaC1₂,20 μl 0.1 M spermidine. Each reagent is added sequentially to the gold particle suspension, while maintained on the multi-tube vortexer. The final mixture was sonicated briefly and allowed to incubate under constant vortexing for 10 minutes. After the precipitation period, the tubes are centrifuged briefly, liquid removed, washed with 250 μ 100% ethanol, and centrifuged for 30 seconds. Again the liquid is removed, and 30 μ 100% ethanol is added to the final gold particle slurry. For particle gun bombardment, the gold/DNA particle prep is briefly sonicated and 5 μis spotted onto the center of each macrocarrier and allowed to dry about 2 minutes before bombardment.

The sample plates are bombarded at level #4 using a DuPont biolistics particle gun. All samples receive a single shot at 650 PSI, with a maximum of six aliquots taken from each tube of prepared particles/DNA., expression cassettes containing the ZmAxig1-driven LEC1 cDNA are then co-introduced into the scutella of these embryos, along with an anthocyanin expression cassette containing the R gene under the control of the maize lec1 promoter and the C1 gene under the control of the ln2-2 inducible promoter (U.S. Pat. No. 5,364,780). As a negative control, embryos are bombarded with the same anthocyanin expression cassette without the LEC1 expression cassette. One to 18 hours following bombardment, embryos are transferred back to 560P culture medium an N6-based medium containing Eriksson's vitamins, thiamine, sucrose, 2,4-D (2 mg/L), and silver nitrate and incubated in the dark at 26° C. Cultures are allowed to grow without selection for four weeks. After four weeks the callus is transferred to a hormone free MS medium for plant regeneration containing safener, an inducer of the ln2-2-2 promoter. During maturation the Lec1 promoter will be activated turning on R expression and the safener will induce C1 expression. Red transformed embryos can then be picked and transferred to germination medium. The resulting plantlets will be green. The plants can then be transferred to the greenhouse and grown to maturity. After pollination the plants can be sprayed with the safener inducer to induce pigmentation in the T1 embryos.

### Example 2

Using nos:Lec1or ZmAxig1:lec1with an inducible CRC expression cassette to recover transformants in the absence of herbicides or antibiotics.

To determine if Lec1 driven by a weak constitutive promoter could be used with an inducible promoter driving CRC to recover transformants the following experiment was performed. Immature High type II embryos (harvested 10 days after pollination) were excised and cultured on 560P(see example 1) medium for 5-6 days. These embryos were then moved to high osmotic 560Y (see example 1) medium to prepare them for particle gun bombardment. Embryos were then shot as described above using a 1:1 mixture of nos:LEC1 and ln2-2:CRC. The ln2-2 promoter is induced by safener and auxins and thus is induced by the 2,4-D in the culture medium. Following particle gun bombardment these embryos were moved to 560P culture medium and allowed to callus without chemical selection. The nos:lec1ln2-2:CRC treatment embryos were maintained on 560P medium. After 2-3weeks transformed multicellular red callus clumps were visually identified on the nos:lec1/ln2-2:CRC treatment callusing and these clumps were moved to fresh 560P medium. Transformation frequencies were tabulated after 10 weeks of culture. Starting with 125 immature embryos a total of 33 events were recovered from the CRC visually selected treatment. On regeneration medium (no 2,4-D) the ln2-2 promoter is no longer induced and most of the resulting somatic embryos and plantlets did not express CRC and appeared normal. Most of these developed normally but a few showed anthocyanin expression in the tassels or leaves.

Variations of the above experiment were conducted to compare visual anthocyanin selection using ln2-2:CRC alone or in combination with lecd driven by an inducible promoter (ZmAxig1). Embryos were shot as described above using either ln2-2:CRC alone (ln2-2:CRC:pinll) or ln2-2:CRC with Axig1:LEC1 (Axig1:LEC1:pinll). Colonies were visually selected for anthocyanin expression. As shown in Table 1 below, very few transformants were recovered using lN2-2:CRC without LEC1.

[image]

### Example 3

Use of ZmAxig1::LEC1 to provide a growth advantage with either embryo specific CRC expression (Lec1:CRC) or inducible CRC expression (ln2-2:CRC) to recover transformants without the use of chemical selection.

An experiment was conducted to determine if the growth advantage conferred by Lec1 could be used to recover transformants in the absence of chemical selection. In this experiment, all of the transforming DNA components were derived from maize (with the exception of the control treatment). High type 11 embryos were bombarded with ZmAxig1::LEC1 (see PCT US 01/22169 and WO 00/28058) to confer a growth advantage along with either CRC (a fusion between the maize transcriptional activators C1 and R that activate the anthocyanin pathway see Bruce, W., Folkerts, O., Garnaat, C., Crasta, O., Roth, B., and Bowen, B., 2000, Plant Cell 12:65-79) driven by the LEC1 promoter (U.S. Ser. No. 09/718,754 filed Nov. 22, 2000) or CRC driven by the ln2-2 promoter. The LEC1 promoter is turned on during mid-embryo development thus the LEC1:CRC construct can be used to visually identify transformed embryos based on the red color due to CRC expression that can be observed once the cultures are moved to regeneration medium. In contrast CRC expression can be observed on maintenance medium when driven by the ln2-2 promoter.

Immature embryos (harvested 9-10 days after pollination) were precultured for 5-7 days then transformed using a particle gun as described in example 1. Embryos were cultured for two weeks without selection on callus maintenance medium. At this time all of the embryos that failed to initiate callus were discarded and the remaining callusing embryos were transferred to fresh callus maintenance medium. Six weeks after bombardment transformed colonies were visually selected from the ln2-2:CRC and were transferred to hormone free regeneration medium. At this time the callusing embryos from the Lec1:CRC treatment were also moved to hormone free MS regeneration medium to visualize transformants. After two weeks on regeneration medium red transformed embryos were identified in the lec1:CRC treatment and subcultured onto fresh regeneration medium. Transformation frequencies were tabulated and plants were regenerated and sent to the greenhouse. In the ln2-2:CRC treatment a total of 101 events were recovered from 193 responsive embryos (215 starting embryos) to obtain a transformation frequency of 52.3%. In the Lec1:CRC treatment a total of 39 events were recovered from 187 responsive embryos (215 starting embryos) to obtain a transformation frequency of 20.8%. Plants were regenerated from the CRC treatments and sent to the greenhouse. Although the embryos transformed with Lec1:CRC were red, most of the regenerating plantlets were green (since the LEC1:CRC construct does not express in vegetative plant tissues). Most of the plants from both CRC treatments developed normally.

What is claimed is

1. A method for identifying transformed monocot cells comprising:
a) introducing an anthocyanin polynucleotide into a target monocot cell, wherein the anthocyanin polynucleotide is operably linked to a promoter capable of expression in the transformed monocot cell,
b) providing an isolated Lec1 protein or isolated Lec1 polynucleotide, wherein the isolated Lec1 polynucleotide is operably linked to a promoter capable of expression in the transformed monocot cell, and
c) identifying transformed cells.

2. The method of claim 1 further comprising a polynucleotide of interest operably linked to a promoter capable of expression in the transformed monocot cell.

3. The method of claim 1, wherein the anthocyanin polynucleotide is operably linked to a constitutive promoter.

4. The method of claim 1, wherein the anthocyanin polynucleotide is operably linked to an inducible promoter.

5. The method of claim 4, wherein the inducible promoter is an ln2-2 promoter or an Axig1 promoter.

6. The method of claim 1, wherein the anthocyanin polynucleotide is operably linked to a tissue specific promoter.

7. The method of claim 6, wherein the tissue specific promoter is a LEC1 promoter.

8. The method of claim 6, wherein the tissue specific promoter is also inducible.

9. The method of claim 8, wherein the tissue specific promoter is an Axig1 promoter.

10. The method of claim 1, wherein the isolated Lec1 polynucleotide is operably linked to an inducible promoter.

11. The method of claim 1, wherein the isolated Lec1 polynucleotide is operably linked to a constitutive promoter.

12. The method of claim 1, wherein the isolated Lec1 polynucleotide is operably linked to a tissue specific promoter.

13. The method of claim 12, wherein the tissue specific promoter is also inducible.

14. The method of claim 13, wherein the tissue specific promoter is an Axig1 promoter.

15. The method of claim 1, wherein the anthocyanin polynucleotide is R, C1, or CRC.

16. An expression cassette comprising an anthocyanin polynucleotide and a Lec1 polynucleotide, wherein each polynucleotide is operably linked to a promoter capable of expression in a monocot plant cell, wherein each promoter can be the same or different.

17. The expression cassette of claim 16 further comprising a polynucleotide of interest.

18. A monocot plant cell comprising an expression cassette comprising an anthocyanin polynucleotide and a Lec1 polynucleotide, wherein each polynucleotide is operably linked to a promoter capable of expression in the monocot plant cell, wherein each promoter can be the same or different.

19. A monocot plant part comprising an expression cassette comprising an anthocyanin polynucleotide and a Lec1 polynucleotide, wherein each polynucleotide is operably linked to a promoter capable of expression in the monocot plant cell, wherein each promoter can be the same or different.

20. A monocot plant comprising an expression cassette comprising an anthocyanin polynucleotide and a Lec1 polynucleotide, wherein each polynucleotide is operably linked to a promoter capable of expression in the monocot plant cell, wherein each promoter can be the same or different.

21. A method for identifying transformed plant cells comprising:
a) introducing a polynucleotide encoding a visual marker into a target plant cell, wherein the polynucleotide encoding the visual marker is operably linked to a tissue specific promoter or inducible promoter capable of expression in the transformed plant cell,
b) providing an isolated growth stimulation protein or an isolated growth stimulation polynucleotide, wherein the isolated growth stimulation polynucleotide is operably linked to a promoter capable of expression in a plant, and
c) identifying transformed plant cells.

22. The method of claim 21 further comprising a polynucleotide of interest operably linked to a promoter capable of expression in the transformed monocot cell.

23. The method of claim 21, wherein the inducible promoter is an ln2-2 promoter or an Axig1 promoter.

24. The method of claim 21, wherein the tissue specific promoter is a LEC1 promoter.

25. The method of claim 21, wherein the visual marker is an anthocyanin polypeptide.

26. The method of claim 21, wherein the visual marker is a fluorescent protein.

27. The method of claim 21, wherein the growth stimulation polynucleotide is a Lec1 polynucleotide.

28. The method of claim 27, wherein the Lec1 polynucleotide is operably linked to a constitutive, a tissue specific, or an inducible promoter.

29. The method of claim 28, wherein the visual marker is an anthocyanin polypeptide.

30. The method of claim 28, wherein the visual marker is fluorescent protein.

31. An expression cassette comprising a polynucleotide encoding a visual marker and a growth stimulation polynucleotide, wherein the polynucleotide encoding the growth stimulation polynucleotide is operably linked to a promoter capable of expression in a plant cell, wherein the polynucleotide encoding the visual marker is operably linked to a tissue specific or inducible promoter capable of expression in a plant cell.

32. The expression cassette of claim 31 further comprising a polynucleotide of interest.

33. A plant cell comprising an expression cassette comprising a polynucleotide encoding a visual marker and a growth stimulation polynucleotide, wherein the polynucleotide encoding the growth stimulation polynucleotide is operably linked to a promoter capable of expression in the plant cell, wherein the polynucleotide encoding the visual marker is operably linked to a tissue specific or inducible promoter capable of expression in a plant cell.

34. A plant part comprising an expression cassette comprising a polynucleotide encoding a visual marker and a growth stimulation polynucleotide, wherein the polynucleotide encoding the growth stimulation polynucleotide is operably linked to a promoter capable of expression in the plant cell, wherein the polynucleotide encoding the visual marker is operably linked to a tissue specific or inducible promoter capable of expression in a plant cell.

35. A plant comprising an expression cassette comprising a polynucleotide encoding a visual marker and a growth stimulation polynucleotide, wherein the polynucleotide encoding the growth stimulation polynucleotide is operably linked to a promoter capable of expression in the plant cell, wherein the polynucleotide encoding the visual marker is operably linked to a tissue specific or inducible promoter capable of expression in a plant cell.

36. A method for regulating anthocyanin production comprising introducing into a monocot cell two or more complementary anthocyanin polynucleotides, wherein each complementary anthocyanin polynucleotide is operably linked to a different promoter capable of expression in a monocot plant cell and having different expression patterns that overlap either temporally and/or in a tissue specific manner.

37. The method of claim 36, wherein one or more of the promoters is a constitutive promoter.

38. The method of claim 36, wherein one or more of the promoters is an inducible promoter.

39. The method of claim 38, wherein the inducible promoter is an ln2-2 promoter or an Axig11 promoter.

40. The method of claim 36, wherein one or more of the promoters is a tissue specific promoter.

41. The method of claim 40, wherein the tissue specific promoter is a LEC1 promoter.

42. An expression cassette comprising two or more complementary anthocyanin polynucleotides, wherein each complementary anthocyanin polynucleotide is operably linked to a different promoter capable of expression in a monocot plant cell and having different expression patterns that overlap either temporally and/or in a tissue specific manner.

43. The expression cassette of claim 42 further comprising a polynucleotide of interest.

44. A plant cell comprising an expression cassette comprising two or more complementary anthocyanin polynucleotides, wherein each complementary anthocyanin polynucleotide is operably linked to a different promoter capable of expression in a monocot plant cell and having different expression patterns that overlap either temporally and/or in a tissue specific manner.

45. A plant part comprising an expression cassette comprising two or more complementary anthocyanin polynucleotides, wherein each complementary anthocyanin polynucleotide is operably linked to a different promoter capable of expression in a monocot plant cell and having different expression patterns that overlap either temporally and/or in a tissue specific manner.

46. A plant comprising an expression cassette comprising two or more complementary anthocyanin polynucleotides, wherein each complementary anthocyanin polynucleotide is operably linked to a different promoter capable of expression in a monocot plant cell and having different expression patterns that overlap either temporally and/or in a tissue specific manner.

47. A method for identifying transformed monocot cells comprising:
a) introducing two or more complementary anthocyanin polynucleotides, wherein each complementary anthocyanin polynucleotide is operably linked to a different promoter capable of expression in a monocot plant cell and having different expression patterns that overlap either temporally and/or in a tissue specific manner,
b) providing an isolated Lec1 protein or an isolated Lec1 polynucleotide, wherein the isolated Lec1 polynucleotide is operably linked to a promoter capable of expression in the transformed monocot cell, and,
c) identifying transformed cells.

48. The method of claim 47, wherein one or more of the promoters is a constitutive promoter.

49. The method of claim 47, wherein one or more of the promoters is an inducible promoter.

50. The method of claim 49, wherein the inducible promoter is an ln2-2 promoter.

51. The method of claim 49, wherein the inducible promoter an Axig1 promoter.

52. The method of claim 47, wherein one or more of the promoters is a tissue specific promoter.

53. The method of claim 52, wherein the tissue specific promoter is a LEC1 promoter.

54. An expression cassette comprising a Lec1 polynucleotide and two or more complementary anthocyanin polynucleotides, wherein each complementary anthocyanin polynucleotide is operably linked to a different promoter capable of expression in a monocot plant cell and having different expression patterns that overlap either temporally and/or in a tissue specific manner, and wherein the Lec1 polynucleotide is operably linked to a promoter capable of expression in a monocot plant cell.

55. The expression cassette of claim 54 further comprising a polynucleotide of interest operably linked to a promoter capable of expression in a monocot plant cell.

56. A plant cell comprising an expression cassette comprising a Lec1 polynucleotide and two or more complementary anthocyanin polynucleotides, wherein each complementary anthocyanin polynucleotide is operably linked to a different promoter capable of expression in a monocot plant cell and having different expression patterns that overlap either temporally and/or in a tissue specific manner, and wherein the Lec1 polynucleotide is operably linked to a promoter capable of expression in a monocot plant cell.

57. A plant part comprising an expression cassette a Lec1 polynucleotide and two or more complementary anthocyanin polynucleotides, wherein each complementary anthocyanin polynucleotide is operably linked to a different promoter capable of expression in a monocot plant cell and having different expression patterns that overlap either temporally and/or in a tissue specific manner, and wherein the Lec1 polynucleotide is operably linked to a promoter capable of expression in a monocot plant cell.

58. A plant comprising an expression cassette comprising a Lec1 polynucleotide and two or more complementary anthocyanin polynucleotides, wherein each complementary anthocyanin polynucleotide is operably linked to a different promoter capable of expression in a monocot plant cell and having different expression patterns that overlap either temporally and/or in a tissue specific manner, and wherein the Lec1 polynucleotide is operably linked to a promoter capable of expression in a monocot plant cell.

59. A method for identifying transformed plant cells comprising:
a) introducing an isolated plant polynucleotide encoding a visual marker into a target plant cell, wherein the polynucleotide encoding the visual marker is operably linked to a promoter capable of expression in the transformed plant cell,
b) providing an isolated plant growth stimulation protein or an isolated plant growth stimulation polynucleotide, wherein the isolated plant growth stimulation polynucleotide is operably linked to a promoter capable of expression in. the transformed plant cell, and
c) identifying transformed plant cells.

60. The method of claim 59, wherein each promoter is isolated from a preselected plant.

61. The method of claim 59, wherein the target plant cell is from a monocot or a dicot.

62. The method of claim 61, wherein the target plant cell is from maize or soybean.

63. The method of claim 59, wherein the promoters, the growth stimulation polynucleotide, and the visual marker polynucleotide are isolated from a plant of the target plant cell.
